(19) United States
(12) Patent Application Publication
(10) Pub. No.: US 2011/0202052 A1
(43) Pub. Date: Aug. 18, 2011

(54) System for treating benign prostatic hyperplasia

(76) Inventors: Daniel Gelbart, Vancouver (CA); Lindsay S. Machan, Vancouver (CA)

(21) Appl. No.: 12/658,374

(22) Filed: Feb. 12, 2010

(51) Int. Cl. A61B 18/18

(52) U.S. Cl. 606/41

(57) ABSTRACT

The disclosed system improves urine flow by increasing the inside diameter of the urethra going through the prostate by eroding the urethral wall, rather than by reducing the prostate volume. This is done by a specially designed IRE electrode, which limits the penetration depth of the electric field to the urethral wall.

### FIELD OF THE INVENTION

The invention is in the medical field and in particular in the area of Benign Prostatic Hyperplasia, commonly known as BPH.

### BACKGROUND OF THE INVENTION

PBH, or the benign enlargement of the prostate in men starting in their mid-forties is very well known and common. By age 80 about 80% of men have signs of this condition. It can cause distressing urination symptoms as the enlarged prostate presses against the urethra and reduces, or even fully blocks, urine flow. Current treatment methods are mainly by drugs, such as Finasteride (Proscar), by surgery to reduce prostate volume and by ablation, including Transurethral Needle Ablation (TUNA). Both surgery and ablation are delicate processes that can result in incontinence and impotence in many cases. The drugs can only improve the condition by a modest amount. Recently new treatment option appeared, such as High Intensity Focused Ultrasound (HIFU) and Irreversible Electroporation (IRE). It is believed that IRE kills the tissue cells by using an electric field (a few volts per cell) to induce microscopic aquatic pores (“electropores”) in the lipid cell membranes. At low levels the effect is reversible and the cells survive. At higher levels it is believed that the pores are permanent and the cause of cell death is chemical imbalance in the cell resulting from fluid communication with the extra cellular environment. The number and size of the pores depends on the electric field used and pulse duration. Typically a DC voltage is used with a value of volts to kilovolts (higher electrode spacing using the higher voltages) and pulse durations in the range of microseconds to milliseconds. IRE has been known for many years but was used inside the human body only recently. Since the exact mechanism of IRE is still being studied, the invention is not limited to the particular explanation given here. The use of IRE in the human body is described in detail is U.S. Pat. No. 6,994,706 and in U.S. patent application Nos. 20070043345 and 20060217703. All these three documents are hereby incorporated by reference in their entirety. FIG. 9 in U.S. application No. 20060217703 disclosed using IRE in a transurethral probe. It is reproduced as FIG. 1 in this application. A catheter 1 has an electrode 2 insulated by layer 3 and connected to a DC pulse source 4 to generate an electric field 6 penetrating the prostate 11. The return of the current can be through ground 5 (FIG. 1-A) or through a second electrode (FIG. 1-B). The electrodes on this probe are widely spaced in order to have the field penetrate into the prostate tissue, causing cell necrosis induced by IRE, and later on shrinking of the prostate. This is similar to the use of RF or microwave to induce necrosis in the prostate cells. It is known from electric field theory that the depth of penetration is on the order of the electrode spacing, for larger distances the field drops very fast, approximately as the third power of the distance. One problem common to all methods reducing the prostate volume, either by direct incision or by TUNA or IRE, is that a large number of cells are removed or killed at once, causing post procedure swelling and requiring using a urethral catheter for an extended period of time, as expected from any trauma to an organ in the urinary tract.

### SUMMARY OF THE DISCLOSURE

The disclosed system improves urine flow by increasing the inside diameter of the urethra going through the prostate by eroding the urethral wall, rather than by reducing the prostate volume. This is done by taking advantage of a unique property of IRE: After IRE treatment not all cells die at the same time; the outer cells die first, as they communicate with the environment. The inner cells, even when perforated by pores, communicate with similar cells and therefore the cell balance does not change rapidly. This causes the necrosis to happen layer by layer, even if all cells were perforated at the same time. This can be viewed as an “extended action” necrosis. A single treatment will cause slow necrosis that can be extended over many days and possible even weeks. Such a slow necrosis, combines with “layer by layer” disposal of the dead cells can be used for a trauma free ablation. This is particularly important when treating BPH. Unlike prior art, which penetrates deep into the prostate and attempts to kill as many prostate cells as possible, the invention limits the cell killing to the urethra and sometimes also to the tissue immediately adjacent to the urethra but controls the depth of penetration in order not to affect the bulk of the prostate, as well as sensitive nerves and blood vessels.

In order to limit the penetration to a shallow depth, a large number of closely spaced electrodes are used, connected together as two groups: positive electrodes and negative electrodes. The positive electrodes are interleaved with the negative electrodes at a dense pitch, in order to create a strong field with a shallow penetration. The electrodes can be a pair of long interleaved spirals, creating multiple electrodes, or discrete small electrodes. A major difference between the invention and prior art is that while prior art attempts to penetrate deep into the prostate by inserting an electrode or a single electrode pair in the urethra, the present invention attempts to have minimal or no penetration into the prostate by using a large number of closely spaced electrodes. The electrode spacing and pulse voltage can be chosen to affect mainly the inside of the urethra.

### BRIEF DESCRIPTION OF THE DRAWINGS

FIGS. 1-A and 1-B are a longitudinal sections of prior art catheters designed to induce IRE in the prostate trough the urethra.

FIG. 2 is a general view of a catheter designed to induce shallow IRE in the urethra using spiral electrodes.

FIG. 3 is a general view of a catheter designed to induce shallow IRE in the urethra using discrete electrodes connected in two groups.

FIG. 4 is a longitudinal section of a catheter using closely spaced electrodes inside the urethra.

FIG. 5 is an enlarged view of a few layers of cells affected by IRE in the urethral wall.

FIG. 6 is a section through the male body depicting the insertion of the catheter into the urethra passing through the prostate.

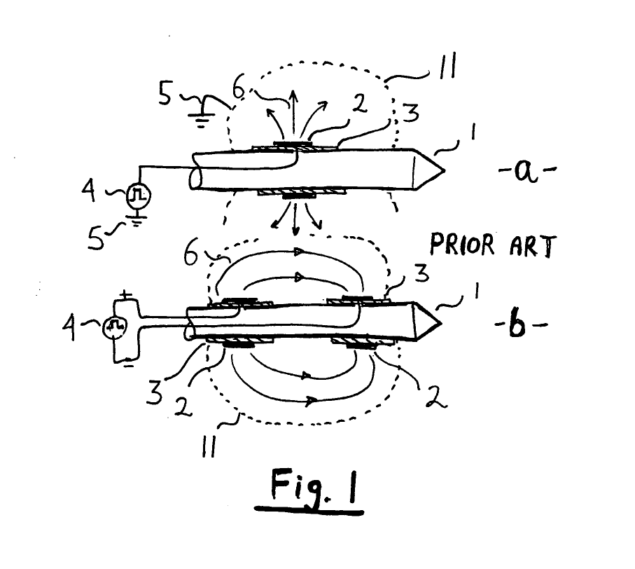

### DETAILED DISCLOSURE

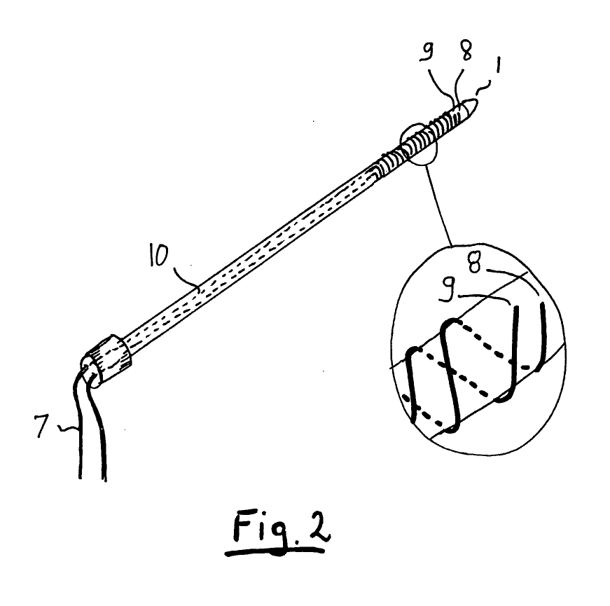

Referring now to FIG. 2, a flexible catheter 1, typically made of polymeric material used to make urethral catheters, has a plurality of closely spaced electrode. One method of generating such an electrode array is by having two interleaved spiral conductors, 8 and 9, (similar to a two-start screw thread). The electrodes 8 and 9 are connected to the external leads 7 via insulated conductors 10. The complete unit can be molded as a single assembly.

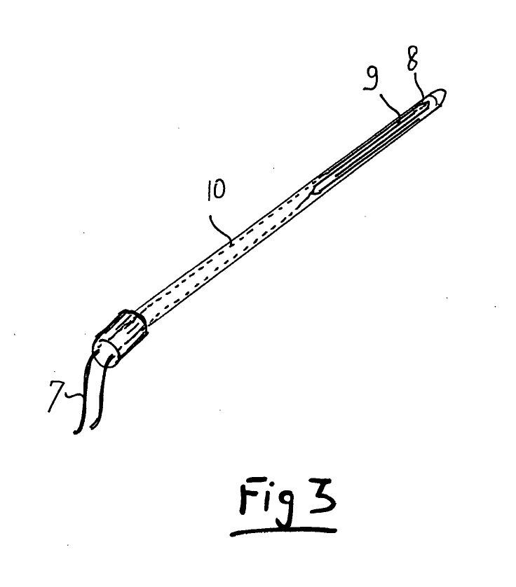

FIG. 3 shows a closely spaced electrode array made by connecting in parallel a number of discrete electrodes 8 and 9. Many other patterns of electrodes can be used, with particular shapes chosen to direct the field and control the depth of penetration according to the well known theory of electromagnetic fields.

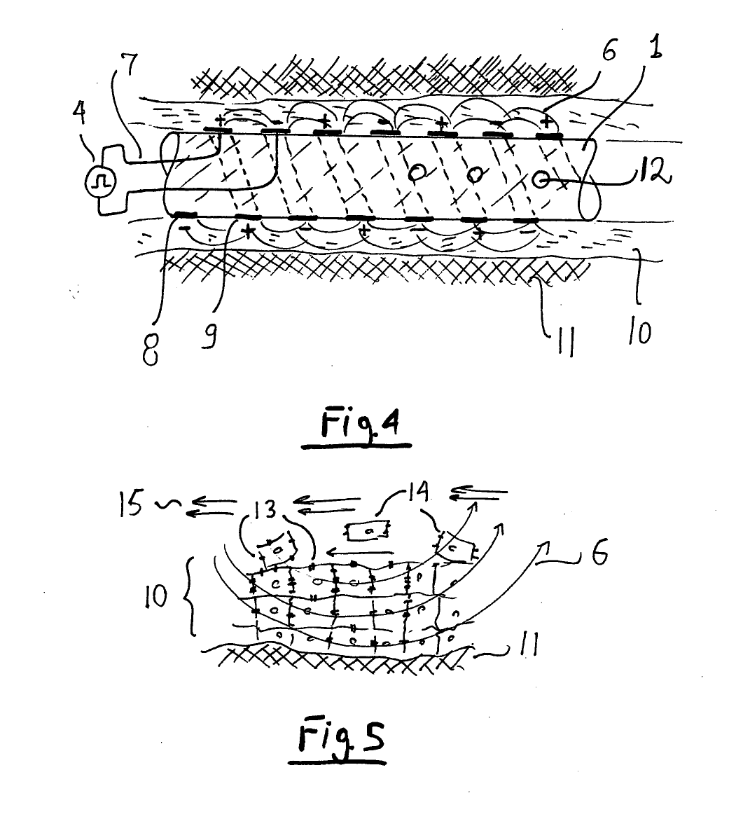

FIG. 4 shows a longitudinal section of a catheter according to the disclosure inserted into the urethra 10 passing through the prostate gland 11. The field 6 is created between multiple electrode pairs 8 and 9 and the penetration into the urethra is of the same order as the electrode spacing, typically in the range of 0.1 mm to a few mm. A preferred range for electrode spacing is from 0.2 mm to 2 mm. The plurality of electrodes 8 and 9 are created by winding two interleaved conductors in a spiral around catheter 1. In areas the urethral wall is thin the field will penetrate slightly beyond the urethral wall into the prostate, but this condition is not detrimental to the procedure. The purpose of the procedure is to enlarge the inside diameter of the urethra by gradually removing tissue from the inside of the urethra, layer by layer. It is well known that the urine will flow through the prostate even if the urethra is damaged or sections removed. A new urethral wall will be formed slowly. Since the electrode pitch is small the voltages used are substantially lower than typically used in IRE: from tens to hundreds of volts instead of hundreds to thousands of volts. The pulse durations are similar to those used in IRE. Catheter 1 can be made hollow and irrigation holes 12 can be added. Such holes can be used to deliver a saline solution for cooling as well as medication to assist the procedure such as for pain reduction, muscle contraction reduction and lubrication. The insertion of catheters into the urethra is a standard medical procedure. After applying a series of DC pulses by generator 4 the catheter is removed and the cells start dying.

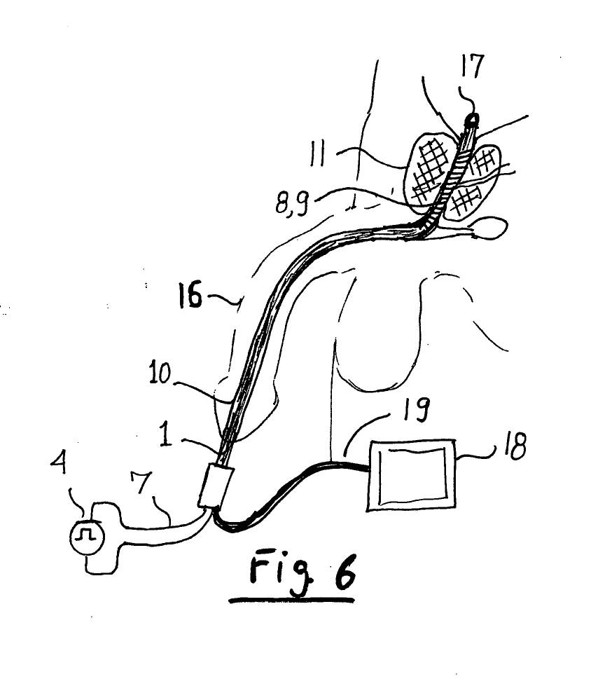

FIG. 5 is a simplified view of the cell necrosis process. The urethral wall 10 is made up of many layers of cells perforated by the electric field 6. Pores 13 allowing the inside of the cells to communicate with the outside. As long as a cell is located in an inner layer, the outside of the cell comprises of other perforated cell and the change in the chemical balance of the cell is slow. In the outer layers 14 the outside environment, typically urine, is very different than the outside environment for the inside cells. Outer layer cells 14 die rapidly and are flushed out by the urine stream 15, exposing a new layer of cells to the urine. The urethral wall 10 s getting progressively thinner, increasing the inside diameter and urine flow. The rate the cells are dying can be controlled by the number of pores, which is controlled by the voltage used. The DC pulse generator for IRE is a standard commercial device, such as NanoKnife™ made by AngioDynamics (www.angiodynamics.com). As in RF ablation, the impedance of the tissue can be monitored during the process and the process terminated when the correct impedance is reached. Because of the gradual necrosis action swelling, trauma and catheter used is minimized and possibly completely eliminates. The rate of necrosis can be controlled by the voltage used as well as catheter electrode design. The complete procedure can be done in a few minutes by inserting the catheter via the penis 16 as shown in FIG. 6. The location of the electrodes 8 and 9 in the correct area of the prostate can be done by an endoscopic camera 17 connected to a display 18 via a fiber optic (or electric) cable 19. In general ablation by IRE is less damaging to nerves and blood vessels than RF ablation, microwave ablation, laser ablation or other ablation modes. This is due to the lower electrical conductivity (for DC currents) of the outer coating of nerves and, to some degree, blood vessels. This advantage is further amplified by the invention: the very shallow penetration of the electric field produced according the invention falls off quickly before reaching the nerve and blood vessels in the prostate. This should greatly reduce the incidence of impotence and incontinence present in today's procedures. While the preferred embodiment is for the treatment of BPH the invention can be used to increase the diameter of other lumens and ducts inside the body, as well as for general removal of a thin layer of tissue with minimal damage to underlying tissue and minimum trauma.

1. A system for improving the flow of urine by enlarging the inside diameter of the urethra, said enlarging done by a shallow penetration IRE system.

2. A system for treating BPH by using shallow penetration IRE system to erode the inside wall of the urethra.

3. A catheter for treating BPH by eroding the inside wall of the urethra, said catheter being inserted inside the urethra and causing a shallow layer of cells inside the urethra to die.

4. A catheter as in claim 3 wherein the cells inside the urethra are made to die by the effect of a shallow electric field.

5. A system as in claim 1 wherein said enlarging is done by inserting a catheter into the urethra, said catheter having electrodes limiting the penetration of the electric field to less than 2 mm.

6. A system as in claim 1 wherein said shallow penetration is done by limiting the distance between the IRE electrodes to less than 2 mm.

7. A system as in claim 3 wherein said shallow layer is less than 2 mm deep.

8. A system as in claim 3 wherein said catheter has electrodes in the form of two spirals.

9. A system as in claim 3 wherein said catheter has electrodes in the form of longitudinal lines.

10. A system as in claim 3 wherein said catheter has a lumen for providing irrigation during said erosion.

11. A system as in claim 1 wherein said shallow penetration is limited to the urethral wall.
